(19) United States
(12) Patent Application Publication
(10) Pub. No.: US 2023/0241233 A1
(43) Pub. Date: Aug. 3, 2023

(54) ORAL PHARMACEUTICAL COMPOSITIONS COMPRISING LIPID CONJUGATES

(71) Applicant: UNIVERSITAT HEIDELBERG, Heidelberg (DE)

(72) Inventors: Max SAUTER, Heidelberg (DE); Walter-Emil HAEFELI, Neckargemünd (DE); Philipp UHL, Heidelberg (DE)

(73) Assignee: UNIVERSITAT HEIDELBERG, Heidelberg (DE)

(21) Appl. No.: 18/015,613

(22) PCT Filed: Jul. 14, 2021
(86) PCT No.: PCT/EP2021/069648

§ 371 Date: Jan. 11, 2023

(30) Foreign Application Priority Data
Jul. 14, 2020 (EP) 20185810.7

(51) Int. Cl. A61K 47/64; A61K 47/54; A61K 9/00; A61K 9/107

(52) U.S. Cl. CPC A61K 47/645; A61K 47/64; A61K 47/548; A61K 9/0053; A61K 9/1075

(57) ABSTRACT

The invention relates to oral pharmaceutical compositions of an active agent and a lipid conjugate of a cell penetrating peptide conjugated to a lipid molecule. The conjugated lipid acts as a permeation enhancer for the active agent in the composition. In other words, oral bioavailability of the active agent increases when co-administered together with the lipid conjugate described herein.

The invention relates to oral pharmaceutical compositions of an active agent and a lipid conjugate.

### PRIOR ART

Oral drug delivery is considered as the most advantageous route of drug application, in particular for the treatment of chronic diseases, which demand long-term and repeated drug administration. The oral route offers high drug safety and is widely accepted among patients due to its convenience. Additionally, as sterility is not required for oral drug forms, costs in production, storage and distribution are reduced, which may contribute to health care improvement in third world countries. It is estimated, that 90% of all marketed drug formulations are for oral use.

However, the number of drugs with low oral bioavailability, such as macromolecular drugs like peptides, proteins, and antibodies is steadily increasing. Because of their sizes, macromolecular drugs have particularly low oral bioavailability. In particular, many macromolecular drugs show poor absorption across the gastrointestinal barrier. Thus, such drugs have to be administered subcutaneously or intravenously which increases necessary medical efforts and causes increased costs, decreased patient compliance, and increased risk of complications.

To overcome this problem, different approaches to improve the oral bioavailability have been tested in the past years including nanoparticles, and liposomes. However, conventional liposomal formulations have not been very convincing due to their instability in the gastrointestinal tract.

Nanoparticle and liposome preparation involves multi-step processes requiring special expertise and expensive equipment. Achieving pure, consistent and stable products requires expert level personnel and experience.

There is a need for pharmaceutical compositions that improve bioavailability of active agents with poor mucosal absorption. Manufacture of the compositions, in particular manufacturing under GMP rules, should be as simple as possible, preferably not involving production of nanoparticles or liposomes.

### DESCRIPTION OF THE INVENTION

In an aspect, the invention relates to a pharmaceutical composition for oral administration comprising
a conjugate comprising a cell penetrating peptide conjugated to a lipid, and
an active agent, optionally selected from the group consisting of peptides, polypeptides and proteins,
wherein the composition is essentially free of liposomes.

In another aspect, the invention relates to a pharmaceutical composition comprising
at least one conjugated lipid comprising a cell penetrating peptide conjugated to a lipid, such as a phospholipid or fatty acid, and
at least one active agent,
   wherein the amount of conjugated lipid is from 0.1 to 100 mol % relative to the total amount of oily components in the composition.

Optionally, the amount of lipid conjugate may be at least 1.25 mol %, at least 3.0 mol %, greater than 5.0 mol %, at least 10.0 mol %, at least 15.0 mol %, at least 20.0 mol % or at least 25.0 mol % relative to the total amount of oily component in the composition.

In an optional embodiment, the composition is a self-emulsifying drug delivery system (SEDDS), such as a self-microemulsifying or self-nanoemulsifying drug delivery system. SEDDS form droplets in the GI tract. The droplets may protect active agents from enzymatic degradation. SEDDS may comprise considerable amounts of oily components so that the amount of lipid conjugate relative to the total amount of oily components in the composition may be low. Optionally, the amount of lipid conjugate in the pharmaceutical composition may be up to 10.0 mol %, up to 8.0 mol %, up to 6.0 mol %, up to 5.0 mol %, up to 4.0 mol % or up to 3.0 mol % relative to the total amount of oily components. The lipid conjugate may serve two functions in SEDDS, e.g. it may enhance resorption of the active agents, and it may additionally serve as surfactant so that no additional surfactant is needed, or the amount of surfactant may be reduced. Optionally, the amount of certain co-solvents may be reduced, e.g. propylene glycol. Optional compositions comprise at least 50 mol %, at least 75 mol %, at least 85 mol % or at least 90 mol % of lipid conjugate relative to the total amount of oily components in the composition.

The peptide may be conjugated to the lipid directly or indirectly via a covalent bond. In this context, indirect conjugation means that one or more linkers may be positioned between lipid and peptide.

It was found that the conjugated lipid acts as a permeation enhancer for the active agent in the composition. In other words, oral bioavailability of the active agent increases when co-administered together with the lipid conjugate described herein. Without wishing to be bound by this theory, the inventors believe that the lipid conjugate may form micelles, droplets, vesicles or particles (hereinafter collectively referred to as “particle”) that enhance resorption of the active agent in a patient's gastrointestinal tract. It appears that the particles form spontaneously without any need for specialized equipment or expertise. The particles may be present in a liquid pharmaceutical composition or they may form after ingestion of a dosage form by a patient.

The term “particle” means a micelle, vesicle, droplet or particle of colloidal dimensions that exists in equilibrium with the molecules or ions in solution from which it is formed. The particles may form spontaneously, i.e. simply by combining the components. In the context of this description, “colloidal dimension” means a particle size of less than 100 nm, preferably less than 75 nm or less than 50 nm. A particle may form spontaneously. The term “particle” does not include “liposome”. As opposed to liposomes, the pharmaceutical compositions of disclosed herein typically do not contain cholesterol in amounts of more than 1.0 mol % relative to the total amount of the oily component. The amount of cholesterol may even be limited to less than 0.5 mol % or less than 0.1 mol % relative to the total amount of oily component. In certain embodiments, the particles may be larger, e.g. in the case of SEDDS larger particles may be formed spontaneously. Optionally, the particles may have a particle size of larger than 100 nm, e.g. from 100 nm to 200 nm, preferably from 100 nm to 300 nm, from 120 nm to 200 nm or from 120 nm to 180 nm. In an embodiment, the particle size ranges from 20 to 500 nm, from 50 to 400 nm, from 100 to 300 nm or from 120 to 200 nm.

The term “liposome” refers to artificially prepared vesicles composed of lipid bilayers. Liposomes can be used for delivery of APIs due to their unique property of encapsulating a portion of an aqueous solution inside a lipophilic bilayer membrane. Lipophilic compounds can be dissolved in the lipid bilayer, and in this way liposomes can carry both lipophilic and hydrophilic compounds. To deliver the molecules to sites of action, the lipid bilayer can fuse with other bilayers such as cell membranes, thus delivering the liposome contents. In an embodiment, the compositions of this disclosure are essentially free of liposomes, particularly of liposomes having an average particle size of at least 100 nm. “Essentially free of liposomes” means that the number of liposomes within the composition is less than 10% or less than 1% relative to the total number of particles in the composition. The number of particles may be determined by cryo-electron microscopy and counting the particles. In some embodiments, e.g. if the composition is in the form of SEDDS, the composition may be dissolved in simulated gastric fluid using the paddle apparatus (Ph. Eur.) before conducting the cryo-electron microscopy. Preferably, the number of liposomes is less than 5%, less than 3% or less than 1% of the total number of particles in the composition. In an embodiment, the composition contains less than 0.1% by weight of liposomes, preferably less than 0.05% by weight of liposomes relative to the total weight of the composition.

In an embodiment, oral bioavailability of the active agent is increased by at least 50%, at least 100%, at least 150%, at least 200% or at least 250% compared to oral bioavailability of the active agent in the same composition administered to a human without the lipid conjugate. Optionally, the absolute oral bioavailability of the active agent in the composition comprising the conjugate may be at least 0.01%, at least 0.05%, at least 0.1%, at least 0.5%, at least 1.0%, at least 2.5% or at least 3.0%.

The absolute oral bioavailability of the active agent may be less than 10.0%, less than 2.5%, less than 1.0%, less than 0.5%, or less than 0.2% when administered to a human in the composition described herein without the conjugate. In other words, the active agent may be an agent with low absolute oral bioavailability. In the absence of any other indication, references to administration or bioavailability refer to administration to a human and bioavailability after administration to a human. Oral bioavailability can be assessed by oral administration to a human in a fasting state. Fasting state means more than 8 hours after food ingestion, e.g. in the morning before breakfast. Subjects should not ingest any food within 2 hours after administration.

Conjugated Lipids

The expressions “conjugated lipid” and “lipid conjugate” are used interchangeably in this description. The lipid conjugates of this invention may have the following general structure:

L-AG-CPP (Formula I)

In this formula, L represents the lipid, AG represents an optional activating group and/or linker and CPP represents the cell penetrating peptide. The hyphens represent covalent bonds. In other words, the lipid conjugate may comprise the lipid covalently attached to the CPP, wherein the covalent bond may optionally be achieved via an activating group and/or linker, or directly without any activating group or other intermediate group in between.

Preferred lipid conjugates have molecular weights in the range of 1.000 to 10.000 g/mol, preferably from 1.200 to 5.000 g/mol, or from 1.500 to 3.500 g/mol.

Optionally, the lipid conjugate may carry a positive charge and/or the net charge of the lipid conjugate may be positive. A conjugate has a positive net charge, if the number of positive charges is larger than the number of negative charges in the conjugate.

Preferred lipid conjugates are shown in FIGS. 11 and 12. An optional lipid conjugate is the following compound A:

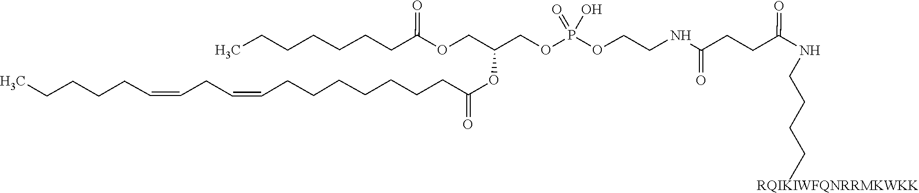

An optional lipid conjugate is the following compound B:

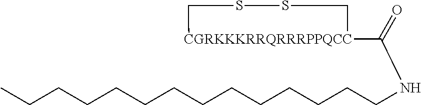

An optional lipid conjugate is the following compound C:

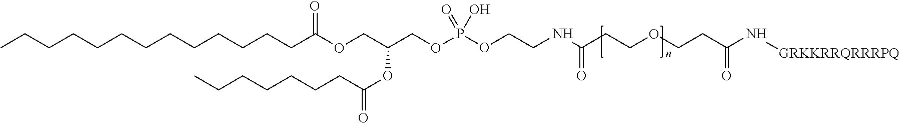

An optional lipid conjugate is the following compound D:

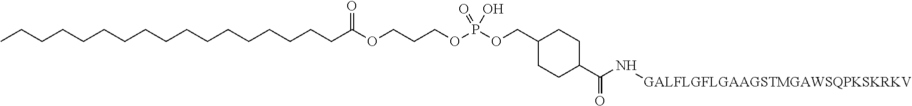

An optional lipid conjugate is the following compound E:

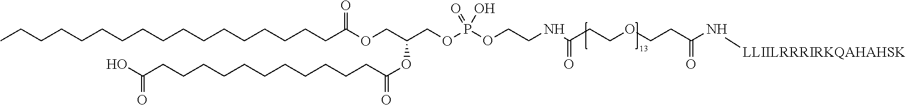

An optional lipid conjugate is the following compound F:

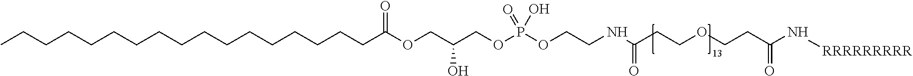

An optional lipid conjugate is the following compound G:

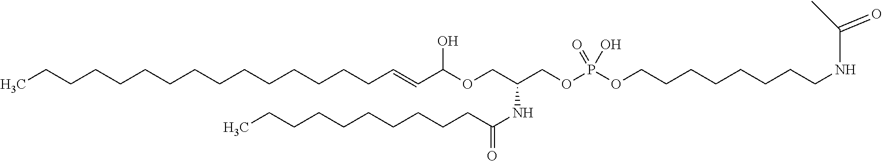 [SMILES: CCCCCCCCCCCCCCC/C=C/C(O)OC[C@H](COP(=O)(O)OCCCCCCCCNC(C)=O)NC(=O)CCCCCCCCCC]

An optional lipid conjugate is the following compound H:

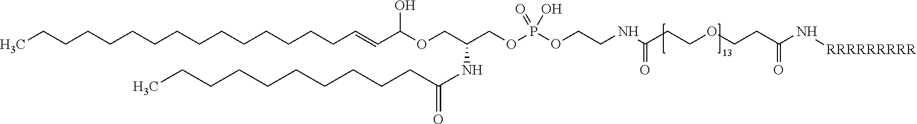

An optional lipid conjugate is the following compound I, wherein R represents arginine and n may be an integer of >3:

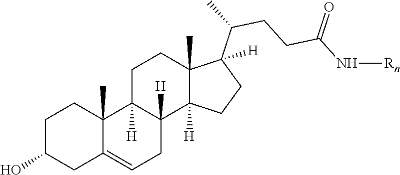

An optional lipid conjugate is the following compound J:

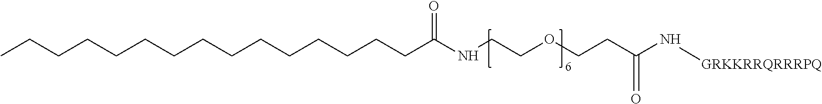

Pharmaceutical Composition

The pharmaceutical composition may be liquid, solid, or semi-solid. The pharmaceutical composition may be in the form of a solution, emulsion, suspension, powder, lyophilisate, granules, pellets, gel, tablet, pill, capsule, effervescent formulation, paste, lozenge, chewing gum, or spray. Liquid compositions may include solvents, such as water, in amounts of at least 50.0 wt %, at least 75.0 wt %, or at least 90.0 wt %. Solid compositions may comprise only limited amounts of solvent, such as less than 50.0 wt %, less than 30.0 wt %, less than 15.0 wt % or less than 5.0 wt %. Optional compositions are free of solvents.

The relative weight amount of lipid conjugates in the pharmaceutical compositions may be in the area of 1 to 25 wt.-% relative to the total weight of the composition. Preferred lower limits include 2 wt.-%, 3 wt.-% or 5 wt.-%. Preferred upper limits include 20 wt.-%, 15 wt.-% or 10 wt.-%.

The weight amount of lipid conjugate in the pharmaceutical composition may be at least as high as the amount of active agent. In an embodiment, the weight amount of lipid conjugate exceeds the amount of active agent by a factor of at least 1.5, at least 2.0, at least 2.5 or at least 3.0. Optionally, the weight amount of lipid conjugate may be limited to about 100 times the amount of active agent, or up to about 50 times, or up to about 25 times or up to about 15 times the amount of active agent.

In an embodiment, the amount of lipid conjugate in the composition is at least 0.1 mg/g relative to the composition. Optionally, the amount may be at least 0.3 mg/g, at least 0.5 mg/g or at least 0.7 mg/g. The amount may be limited to up to 700 mg/g, up to 500 mg/g, up to 300 mg/g, up to 200 mg/g or up to 100 mg/g. In embodiments, the amount of lipid conjugate is up to 70 mg/g, up to 50 mg/g or up to 35 mg/g of the composition. In an exemplary embodiment, the amount is from 0.1 to 700 mg/g, from 0.3 to 300 mg/g, from 0.5 to 100 mg/g or from 0.7 to 35 mg/g. The amount may range up to 1000 mg/g.

In an embodiment of a liquid composition, the amount of lipid in the composition may be selected in a range of from 0.1 to 2.0 mg/ml, or from 0.3 to 1.5 mg/ml or from 0.5 to 1.0 mg/ml. Optionally, the amount is at least 0.7 mg/ml. It was found that these concentrations are best suited for particle formation.

Optionally, the composition comprises an aqueous solution with particles therein, wherein the particles comprise the lipid conjugate. It was found that the lipid conjugates described herein have the capability of forming particles upon contact with aqueous media, such as buffers. The aqueous solution may be a buffer, such as a citrate buffer or a phosphate buffer or a mixture thereof. Because the particles form spontaneously upon mixing the lipid conjugates with aqueous media (self-emulsifying), the difficult preparation of liposomes is not necessary. Optionally, the particles have an average particle size of less than 100 nm, or less than 75 nm, or less than 50 nm, or less than 40 nm or less than 30 nm. The particles may be much smaller than liposomes. Typically, liposomes of the lipids used herein are much larger than 100 nm. It is surprising that the liposomes are not needed to achieve the effect of increased bioavailability. The zeta potential of the particles may be positive, such as at least 1.0 mV, preferably at least 2.0 mV, at least 3.0 mV or at least 4.0 mV. Conventional liposomes typically have negative zeta potentials because of the negative charges of the lipids in their bilayers.

Pharmaceutical compositions of this invention may or may not contain the particles described above. Optional embodiments are in the form of solid compositions without any particles. The inventors hypothesize that upon resuspension of the solid composition in the stomach particles may form after administration. In one embodiment, the pharmaceutical composition is a solid dosage form comprising active agent and lipid conjugate in lyophilized form.

In an embodiment, the invention includes pharmaceutical compositions comprising one or more oily components in an amount of not more than 10.0 wt % relative to the pharmaceutical composition, one or more lipid conjugates in a total amount of at least 25.0 mol % relative to the total amount of oily components, and one or more active agents. The active agent may be a peptide. The pharmaceutical composition may be a solid composition, such as a tablet, or a capsule.

In another embodiment, the invention includes pharmaceutical compositions comprising one or more oily components in a total amount of at least 50.0 wt % relative to the pharmaceutical composition, one or more lipid conjugates in a total amount of at least 1.0 mol % relative to the total amount of oily components, and one or more active agents. The active agent may be a peptide. The amount of cholesterol in the pharmaceutical composition may be limited to not more than 1.0 mol % relative to the total amount of oily component. The pharmaceutical composition may be a liquid composition, such as an emulsion and/or a SEDDS. Optionally, the pharmaceutical composition may be essentially free of nanoparticles and/or essentially free of any particles of more than 100 nm particle size. In this context, “essentially free” means that the content of the mentioned constituents is less than 0.5 wt. %, less than 0.1 wt. % or even less than 0.01 wt. % of the composition.

The pharmaceutical compositions may comprise at least one pharmaceutically acceptable excipient, and/or at least one protease inhibitor, and/or at least one lipase inhibitor. These substances can be incorporated into the dosage form. Preferably, said at least one pharmaceutically acceptable excipient is selected from the group consisting of sorbitan monostearate, tripalmitin, cetyl palmitate, alginate, ethyl oleate, C8 triglycerides, C10 triglycerides, cellulose, disaccharides, monosaccharides, oligosaccharides, magnesium stearate, corn starch, citric acid, tartaric acid, acid salts of amino acids, and combinations thereof. Some of these excipients may form part of or constitute the oily components fraction of the composition. Furthermore, said at least one protease inhibitor is preferably selected from the group consisting of aprotinin, soybean trypsin inhibitor, bacitracin, sodium glycocholate, bestatin, leupeptin, cystatin, camostat mesilate, and combinations thereof. Furthermore, said at least one lipase inhibitor is preferably selected from the group, consisting of orlistat, lipstatin, chitin, chitosan, saponin, flavonoid glycoside, polyphenole, ebelacton A and B, esterastin, valilactone, panclicine, proanthocyanidin, vibralactone, and combinations thereof.

Optionally, the lipid conjugate is not part of a liposome's lipid double layer. Particularly, the cell penetrating peptide may be attached to a compound that is not part of a liposome's lipid double layer. Optionally, the pharmaceutical compositions are free of tetraetherlipids.

Lipids

The lipid may be selected from the group consisting of steroids, fatty acids, fatty alcohols, fatty amines, hydrocarbons with carbon chain lengths of at least eight carbon atoms (e.g. liquid paraffin), phospholipids, sphingolipids, ceramides, glycolipids, etherlipids, polyethers, carotenoids, and glycerides (mono-, di- and/or triglycerides) and combinations thereof. Steroids include compounds having a sterane structure. Steroids include cholesterol and its derivatives. Triglycerides include medium chain triglycerides (e.g. C6 to C12 fatty acids). Mono-, di- or triglycerides and/or fatty acids may be modified, such as PEGylated, ethoxylated, esterified (e.g. with propylene glycol, sorbitol or sorbitan) and/or in salt form. Mono-, di- or triglycerides include vegetable oils.

The lipids in the conjugated lipid may be selected from amphiphilic compounds/surfactants. Optional lipids with surfactant properties may be selected from the group consisting of mono- and/or diglycerides, ethoxylated mono-, di- or triglycerides (e.g. Kolliphor® EL), medium chain mono- and/or diglycerides (e.g. C6 to C12 fatty acids), ethoxylated plant oil (e.g. ethoxylated castor oil), monoglycerides of C12 to C20 saturated or unsaturated fatty acids (e.g. C14 to C18), esters of saturated or unsaturated fatty acids with diols (such as C8 to C20 fatty acids; e.g. 2-hydroxypropyl octanoate; propylene glycol monolaurate), mono- and di-esters esters of polyethylene glycol with medium chain fatty acids (e.g. C6 to 010 fatty acids), sorbitol or sorbitan esters, esters of sorbitol or sorbitan with polyethylene glycol and/or fatty acids (e.g. polysorbates), transesterified ethoxylated vegetable oils (e.g. Labrafil® M1944CS), polyethers (e.g. copolymers of polyalkylene glycols, such as Poloxamer®), salts of fatty acids, cetrimonium bromide, bis(2-ethylhexyl) sulfosuccinate and mixtures thereof.

Examples of lipids suitable for conjugate formation within the concept of this invention are listed in Nardin et al., Successful development of oral SEDDS: screening of excipients from the industrial point of view, Advanced Drug Delivery Reviews 142 (2019) 128-140. The disclosure of Nardin et al. is incorporated by reference as if fully set forth herein. Lipids may include the lipids disclosed in Nardin et al. in Tables 1 and 2; and/or the amphiphilic substances listed in Table 3.

The hydrocarbons preferably comprise at least one activating group for chemical coupling, such as maleimide active ester, amine alcohol, halogenide, thiol, ketone or aldehyde, triple and/or double carbon bonds.

In an embodiment the fatty acids, fatty amines, fatty alcohols and/or hydrocarbons may have carbon chain lengths of from 8 to 24 carbon atoms, preferably from 12 to 20 carbon atoms, or from 16 to 20 carbon atoms. The fatty acids, fatty amines, fatty alcohols and/or hydrocarbons may be saturated or unsaturated. Saturated compounds have the advantage of being chemically more stable than the unsaturated ones.

Preferred lipids include phospholipids and fatty acids.

In an embodiment, the phospholipids may be synthetic, semi-synthetic or natural phospholipids, or combinations thereof. Preferred phospholipids include phosphatidylcholines, phosphatidylethanolamines, phosphatidylinosites, phosphatidylserines, cephalines, phosphatidylglycerols, lysophospholipids, and combinations thereof. Preferred lipids may be selected from 1,2-dioleoyl-snglycero-3-phosphoethanolamine-N-[4-(p-maleimidomethyl)cyclohexane-carboxamide] (sodium salt), 1,2-Dipalmitoyl-sn-Glycero-3-Phosphothioethanol (Sodium Salt), 1,2-dioleoyl-sn-glycero-3-phosphoethanolamine-N-(succinyl) (sodium salt), 1,2-distearoyl-sn-glycero-3-phosphoethanolamine-N-[maleimide(polyethylene glycol)-2000] (ammonium salt), and combinations thereof.

In an embodiment, the lipid is activated. Activation of the lipid may facilitate reaction of the CPP with the first lipid during CPP-lipid conjugate formation. An activated lipid includes lipids that comprise an activating group and/or linker. An activating group may be a group that has a greater reactivity towards the CPP than the lipid without the activating group. Suitable activating groups include activated polymeric groups (molecular weight more than 1.000 g/mol) and small molecule activating groups (molecular weight up to 1.000 g/mol). The reaction product of activated lipid and CPP is a lipid conjugate wherein the lipid and CPP are indirectly, covalently linked. The activating group forms a covalent bond to the CPP.

Activated polymeric groups may be selected from the group consisting of a polymeric part, e.g. polyethylene glycol (PEG), covalently linked to one or more small molecule activating groups selected from a maleimide group (Mal), active esters, such as N-hydroxy succinimide (NHS), tetra fluorophenol (Tfp), and para-nitrophenol esters; amines, alcohols, ketones, aldehydes, thiols, halides, triple and double carbon bonds, and combinations thereof. Thus, an activating group may comprise a polymeric part and a reactive group covalently coupled to each other so that the polymeric part links the reactive group to the lipid. Exemplary activated polymeric groups include SM(PEG)₂₄ (PEGylated, long-chain SMCC crosslinker), SMCC (succinimidyl (N-maleimidomethyl)cyclohexane-1-carboxylate)-linker, 6-maleimido hexanoic acid linker, and combinations thereof. The length of the polymeric part may influence the composition's properties. Accordingly, a preferred PEG polymeric part of an activated polymeric group may have a length of 8 to 50 individual PEG units.

Preferred activated lipids may be selected from the group consisting of (2R)-3-((((4,46-dioxo-46-(2,3,5,6-tetrafluorophenoxy)-7,10,13,16,19,22,25,28,31,34,37,40,43-tridecaoxa-3-azahexatetracontyl)oxy)(hydroxy) phosphoryl)oxy)propane-1,2-diyldistearate (PEG(13)-distearoylphosphatidylethanolamine-tetrafluorophenyl ester); 1,2-dipalmitoyl-sn-glycero-3-phosphoethanolamine-N-[4-(p-maleimidomethyl)cyclo-hexane-carboxamide] (sodium salt), 1,2-dioleoyl-sn-glycero-3-phosphoethanol-amine-N-[4-(p-maleimidomethyl)cyclohexane-carboxamide] (sodium salt), DSPE-PEG(2000) Maleimide (1,2-distearoyl-sn-glycero-3-phosphoethanolamine-N-[maleimide(polyethylene glycol)-2000] (ammonium salt)), Tfp-PEG₁₃-DSPE, Mal-PEG₁₂-DSPE and combinations thereof.

Oily Components

Oily components are lipid and amphiphilic substances comprised in the composition that are not conjugated to a cell penetrating peptide. Oily components may be used in pharmaceutical compositions, e.g. in emulsions, SEDDS or other compositions.

In an embodiment, the oily components include all ingredients of the pharmaceutical composition, except the conjugated lipids, having an n-octanol/water partition coefficient of at least 1.0, at least 2.0 or at least 3.0 at 25° C. Oily components may be the components of the composition, except the conjugated lipids, that are immiscible with water at 25° C. Oily components may be components having saturated or unsaturated carbon chain lengths of more than 6, more than 8 or more than 10 carbon atoms. Optional oily components are modified or unmodified fatty acids.

Oily components may be selected from the group consisting of steroids, fatty acids, fatty alcohols, fatty amines, hydrocarbons with carbon chain lengths of at least eight carbon atoms (e.g. liquid paraffin), phospholipids, sphingolipids, ceramides, glycolipids, etherlipids, polyethers, carotenoids, and glycerides (mono-, di- and/or triglycerides) and combinations thereof. Steroids may include compounds having a sterane structure. Steroids may include cholesterol and its derivatives. Triglycerides include medium chain triglycerides (e.g. C6 to C12 fatty acids). Mono-, di- or triglycerides and/or fatty acids may be modified, such as PEGylated, ethoxylated, esterified (e.g. with propylene glycol, sorbitol or sorbitan) and/or in salt form. Mono-, di- or triglycerides include vegetable oils.

Oily components may include amphiphilic compounds/surfactants. Optional oily components with surfactant properties may include mono- and/or diglycerides, ethoxylated mono-, di- or triglycerides (e.g. Kolliphor® EL), medium chain mono- and/or diglycerides (e.g. C6 to C12 fatty acids), ethoxylated plant oil (e.g. ethoxylated castor oil), monoglycerides of C12 to C20 saturated or unsaturated fatty acids (e.g. C14 to C18), esters of saturated or unsaturated fatty acids with diols (such as C8 to C20 fatty acids; e.g. 2-hydroxypropyl octanoate; propylene glycol monolaurate), mono- and di-esters esters of polyethylene glycol with medium chain fatty acids (e.g. C6 to 010 fatty acids), sorbitol or sorbitan esters, esters of sorbitol or sorbitan with polyethylene glycol and/or fatty acids (e.g. polysorbates), transesterified ethoxylated vegetable oils (e.g. Labrafil® M19440S), polyethers (e.g. copolymers of polyalkylene glycols, such as Poloxamer®), salts of fatty acids, cetrimonium bromide, bis(2-ethylhexyl) sulfosuccinate and mixtures thereof.

Examples of oily components suitable within the concept of this invention are listed in Nardin et al., Successful development of oral SEDDS: screening of excipients from the industrial point of view, Advanced Drug Delivery Reviews 142 (2019) 128-140. The disclosure of Nardin et al. is incorporated by reference as if fully set forth herein. Oily components may include the lipids disclosed in Nardin et al. in Tables 1 and 2; and/or the amphiphilic substances listed in Table 3.

The total amount of oily components in the composition comprises the cumulative amounts of the oily components listed above, in particular of steroids (including cholesterol and its derivatives), fatty acids, fatty alcohols, fatty amines, hydrocarbons with carbon chain lengths of at least eight carbon atoms, phospholipids, sphingolipids, ceramides, glycolipids, etherlipids, polyethers, carotenoids, and glycerides (mono-, di- and/or triglycerides) and combinations thereof, including modified mono-, di- or triglycerides and/or modified fatty acids, such as PEGylated, ethoxylated, esterified (e.g. with propylene glycol, sorbitol or sorbitan) and/or in salt form. Mono, di- or triglycerides include vegetable oils.

The pharmaceutical composition may include oily components in amounts of at least 1.0 wt %, at least 5.0 wt %, at least 10.0 wt %, at least 20.0 wt %, at least 30.0 wt %, at least 40.0 wt % or at least 50.0 wt %. Optionally, the amount of oily components in the composition may be limited to up to 99.0 wt %, up to 95.0 wt %, up to 90.0 wt %, or up to 85.0 wt %.

In certain embodiments with higher amounts of oily components, such as SEDDS, the total amount of oily components in the pharmaceutical composition may be at least 60.0 wt %, at least 70.0 wt % or at least 80.0 wt %.

Alternative embodiments of pharmaceutical compositions with lower amounts of oily components, such as optional types of solid dosage forms, include up to 10.0 wt %, up to 8.0 wt %, up to 6.0 wt % or up to 4.0 wt % of oily components.

Optional embodiments include oily components in amounts of from 1.0 wt % to 99.0 wt %, from 5.0 wt % to 95.0 wt %, or from 10.0 wt % to 90.0 wt %.

In embodiments, the total amount of oily components in the composition is from 0.1 to 100 mol % relative to the total amount of lipid conjugate in the composition. Optionally, the total amount of oily components in the composition is from 5.0 to 100 mol % relative to the total amount of lipid conjugate in the composition. Optionally, the total amount of oily components may be at least 1.25 mol %, at least 3.0 mol %, greater than 5.0 mol %, at least 10.0 mol %, at least 15.0 mol %, at least 20.0 mol % or at least 25.0 mol % relative to the amount of lipid conjugate in the composition. Optional compositions comprise at least 50 mol %, at least 75 mol %, at least 85 mol % or at least 90 mol % of oily components relative to the amount of lipid conjugate in the composition.

The “total lipid content” of the composition is the sum of the proportions of lipid conjugate and oily components.

In certain embodiments, the amount of conjugated lipid is from 0.1 to 100 mol % relative to the total lipid content in the composition. Optionally, the amount of lipid conjugate may be at least 1.25 mol %, at least 3.0 mol %, greater than 5.0 mol %, at least 10.0 mol %, at least 15.0 mol %, at least 20.0 mol % or at least 25.0 mol % relative to the total lipid content in the composition. Optionally, the amount of lipid conjugate in the pharmaceutical composition may be up to 10.0 mol %, up to 8.0 mol %, up to 6.0 mol %, up to 5.0 mol %, up to 4.0 mol % or up to 3.0 mol % relative to the total lipid content. Optional compositions comprise at least 50 mol %, at least 75 mol %, at least 85 mol % or at least 90 mol % of lipid conjugate relative to the total lipid content in the composition.

Further Excipients

The pharmaceutical composition may contain excipients, including solubilizers and solvents.

Solubilizers may include 2-(2-Ethoxyethoxy)ethanol, propylene glycol, glycerol, tetraglycol and combinations thereof. Generally, solubilizers may be present in the pharmaceutical composition in amounts of up to 20.0 wt %, up to 15.0 wt %, or up to 10.0 wt %. Optionally, the amount may be at least 1.0 wt %, or at least 3.0 wt %.

Solvents may include water, dimethyl sulfoxide, ethanol, isopropanole and combinations thereof. The amount of solvent in the pharmaceutical composition may range from 1.0 wt % to 99.0 wt %. Liquid compositions may contain more solvent than semi-solid and solid compositions.

Cell Penetrating Peptides

The cell penetrating peptides (CPPs) in the conjugate may be selected from penetratin, TAT (transactivator of transcription), MAP (model amphiphatic peptide), polyarginines (including R3, R4, R5, R6, R7, R8, R9, R10, R11 and R12), pVEC, transportan, MPG, and combinations thereof. The CPPs may be cyclized or linear, dimerized or un-dimerized. The CPPs may consist of the following sequences, or comprise the following sequences. Penetratin may include SEQ ID NO: 1; TAT may include SEQ ID NO: 2; MAP may include SEQ ID NO: 3; R9 may include SEQ ID NO: 4; pVEC may include SEQ ID NO: 5; transportan may include SEQ ID NO: 6; and/or MPG may include SEQ ID NO: 7. The listed CPPs include functional derivatives and peptide-mimetics of the mentioned sequences. Functional derivatives include CPPs that consist of or comprise the above-mentioned sequences, or sequences having at least 90%, or at least 95% sequence identity therewith. Optional functional derivatives have the sequences disclosed above with up to one, up to two or up to three amino acids replaced by other amino acids. Optionally, the functional derivative may include additional amino acids.

In an embodiment the CPPs used in this invention are positively charged and/or cyclized. Cyclized CPPs have the advantage of being less reactive and more stable than linear CPPs, which is advantageous within the concept of this invention. Cyclic peptides are more stable towards enzymatic cleavage than linear CPPs. As used herein, the term “cyclized” is not to be construed as relating to a peptide having one ring system only, i.e., the present invention is not limited to monocyclic peptides. Accordingly, the present invention includes cyclopeptides wherein two or more ring systems are covalently linked to each other. Furthermore, the cyclopeptides may also comprise amino acids that are not part of the ring system, i.e. the invention includes branched cyclopeptides. Preferably, the cyclopeptides are monocyclic peptides, and more preferably unbranched monocyclic peptides. Further, the CPPs can be composed of L-amino acids, D-amino acids, or mixtures thereof, wherein for linear CPPs, D-amino acids are preferred.

In an embodiment, the CPPs comprise a majority of lysine and/or arginine moieties, which have isoelectric points of around 9.5 and 11, respectively. Due to their additional amino or guanidine group, these two amino acids are positively charged under neutral and even under weakly basic conditions. Accordingly, a CPP mostly comprising moieties of said two specific amino acids is positively charged under neutral and weakly basic conditions as well. Herein, the term “majority” means that at least 30%, preferably at least 50%, more preferably at least 60%, and particularly at least 70% of the amino acids forming the CPP molecule are lysine and/or arginine moieties. Thereby, it is ensured that the CPPs have a positive charge under neutral and weakly basic conditions, i.e., have an isoelectric point of more than 7.0. Therefore, in a specific embodiment of the present invention, the CPPs have an isoelectric point of more than 7.0, preferably of more than 7.5, more preferably of more than 8.0 and particularly preferably of more than 8.5. In this context, the isoelectric point of the CPP is the arithmetic mean of the isoelectric points of the amino acids forming the CPP.

In a specific embodiment of the present invention, the CPPs comprise between 2 to 19, preferably between 3 to 16, more preferably between 4 to 14, and particularly preferably between 6 to 12 arginine moieties as well as one or more moieties selected from the group consisting of tyrosine, threonine, serine, lysine, aspartic acid, glutamic acid, glutamine, asparagine and cysteine. For example, the CPP may comprise nine arginine moieties and one cysteine moiety in a ring system, and are referred to as a cyclic cysteine R9 derivative (such as SEQ ID NO: 8; RRRRRRRRRC). Another preferred example is a cyclopeptide comprising nine arginine moieties and one lysine moiety in the ring system, which is referred to as cyclic R9K derivative (SEQ ID NO: 9; RRRRRRRRRK).

The CPPs of this invention include dimerized CPPs, wherein homo- and heterodimers are within the scope of this disclosure. Dimerization of CPPs can be effected by any means known in the art. In a particular embodiment, CPPs are dimerized via the tripeptide KAK.

The amino acids forming the cyclopeptides are not limited to proteinogenic amino acids. Herein, the amino acids may be selected from any amino acids known in the art, and may include the respective D-enantiomer, L-enantiomer, or any mixture thereof.

CPPs may include peptide-mimetics of the CPPs mentioned above. Peptido-mimetics include depsipeptides and peptoids of the CPPs disclosed herein. A depsipeptide CPP is a CPP wherein at least one peptide bond was replaced by an ester bond.

Active Agent

According to this invention, “peptides” have at least one peptide bond, linking at least two amino acids together. “Polypeptides” have at least eight amino acids. “Proteins” have at least 30 amino acids. This invention is particularly useful for “polypeptides” and “proteins” as active agents. Suitable active agents may be cyclic peptides. Cyclic peptides have improved resistance to gastric fluid. Preferred active agents have excellent stability in gastric and/or intestinal fluids. In an embodiment, the active agent exhibits a degradation half-life in simulated gastric and/or intestinal fluid at 37° C. of at least 10 minutes, preferably at least 20 minutes or at least 30 minutes.

For testing the stability in simulated gastric (comprising pepsin) and intestinal fluid (comprising pancreatin), dissolved peptides can be incubated in fast state simulated gastric fluid or in fast state simulated intestinal fluid (FaSSGF and FaSSIF) prepared according to USP and incubated on a shaker at 37° C. for one hour. Samples can be withdrawn after 5, 10, 15, 30, 45 and 60 min and analyzed by HPLC or LC-MS. From the obtained profile a degradation half-life can be calculated.

Optionally, the active agent is a peptidic active agent. Useful peptidic active agents include peptides and proteins, such as polypeptides having at least six amino acids. Preferred active agents have molecular weights in the range of from 600 to 200.000 g/mol, preferably from 1.000 to 150.000 g/mol, or from 2.000 to 80.000 g/mol. Preferred active agents may be selected from anti-cancer agents (e.g. rituximab, trastuzumab, nivolumab); immune-modulatory agents (e.g. adalimumab, eternacept, cytokines, interferons, glatiramer acetate); hormones such as insulin, GLP-1 analogues such as exenatide and liraglutide, somatostatin and its analogues such as octreotide and pasireotide, hGh, and; glycopeptide antibiotics e.g. vancomycin and daptomycin; peptide drugs for hepatitis treatment such as Myrcludex B; and peptides and antibodies acting on cellular receptors such as glucagon, leuprolide, octreotide, vasopressin, and cetuximab.

Suitable active agents include, without limitation, vancomycin, glatiramer acetate, bulevirtide, octreotide, insulins, and liraglutide, as well as other GLP (glucagon-like peptide)-analogues such as exenatide, lixisenatide, albiglutide, dulaglutide, taspoglutide, and semaglutide, and antibodies (e.g. etanercept; pegfilgrastim; adalimumab, infliximab, rituximab, epoietin alfa, tratuzumab, ranibizumab, beta-interferon, omalizumab). Other examples include pharmaceutically active agents selected from the group consisting of hormones, such as human growth hormone, growth hormone releasing hormone, growth hormone releasing peptide, interferons, colony stimulating factors, interleukins, macrophage activating factor, macrophage peptide, B cell factor, T cell factor, protein A, allergy inhibitor, cell necrosis glycoproteins, immunotoxin, lymphotoxin, tumor necrosis factor, tumor suppressors, metastasis growth factor, alpha-1 antitrypsin, albumin and fragment polypeptides thereof, apolipoprotein-E, erythropoietin, factor VII, factor VIII, factor IX, plasminogen activating factor, urokinase, streptokinase, protein C, C-reactive protein, renin inhibitor, collagenase inhibitor, superoxide dismutase, platelet-derived growth factor, epidermal growth factor, osteogenic growth factor, bone stimulating protein, insulin, atriopeptin, cartilage inducing factor, connective tissue activating factor, follicle stimulating hormone, luteinizing hormone, luteinizing hormone releasing hormone, nerve growth factors, parathyroid hormone, relaxin, secretin, somatomedin, insulin-like growth factor, adrenocortical hormone, glucagon, cholecystokinin, pancreatic polypeptide, gastrin releasing peptide, corticotropin releasing factor, thyroid stimulating hormone, monoclonal or polyclonal antibodies against various viruses, bacteria, or toxins, virus-derived vaccine antigens, cyclosporine, rifampycin, lopinavir, ritonavir, telavancin, oritavancin, dalbavancin, bisphosphonates, itraconazole, danazol, paclitaxel, naproxen, capsaicin, albuterol sulfate, terbutaline sulfate, diphenhydramine hydrochloride, chlorpheniramine maleate, loratidine hydrochloride, fexofenadine hydrochloride, phenylbutazone, nifedipine, carbamazepine, betamethasone, dexamethasone, prednisone, hydrocortisone, 17 beta-estradiol, ketoconazole, mefenamic acid, beclomethasone, alprazolam, midazolam, miconazole, ibuprofen, ketoprofen, prednisolone, methylprednisone, phenytoin, testosterone, flunisolide, diflunisal, budesonide, fluticasone, glucagon-like peptide, C-Peptide, calcitonin, lutenizing hormone, prolactin, adrenocorticotropic hormone, leuprolide, interferon alpha-2b, interferon beta-Ia, sargramostim, aldesleukin, interferon alpha-2a, interferon alpha-n3alpha-proteinase inhibitor, etidronate, nafarelin, chorionic gonadotropin, prostaglandin E2, epoprostenol, acarbose, metformin, desmopressin, cyclodextrin, antibiotics, antifungal drugs, steroids, anti-cancer drugs, analgesics, anti-inflammatory agents, anthelmintics, anti-arrhythmic agents, penicillins, anticoagulants, antidepressants, antidiabetic agents, antiepileptics, antihistamines, antihypertensive agents, antimuscarinic agents, antimycobacterial agents, antineoplastic agents, CNS-active agents, immunosuppressants, antithyroid agents, antiviral agents, anxiolytic sedatives, hypnotics, neuroleptics, astringents, beta-adrenoceptor blocking agents, blood products and substitutes, cardiacinotropic agents, contrast media, corticosteroids, cough suppressants, expectorants, mucolytics, diuretics, dopaminergics, antiparkinsonian agents, haemostatics, immunological agents, lipid regulating agents, muscle relaxants, parasympathomimetics, parathyroid calcitonin, prostaglandins, radiopharmaceuticals, sex hormones, anti-allergic agents, stimulants, anoretics, sympathomimetics, thyroid agents, vasidilators, xanthines, heparins, therapeutic oligonucleotides, somatostatins and analogues thereof, and pharmacologically acceptable organic and inorganic salts or metal complexes thereof. Optionally, the active agent may be selected from pasireotide, bremelanotide, oxytocin, ziconotide, corticorelin, enfuvirtide, eptifibatide, buserelin, goserelin, leuprolide, lanreotide, glatiramer acetate, pentagastrin and combinations thereof. In an embodiment, the pharmaceutical composition is free of nucleic acid active agents, such as DNA and RNA.

Examples of such peptidic active agents are somatostatin receptor agonists such as pasireotide, lanreotide, octreotide or combinations thereof. Another preferred group is the glucagon-like peptide-1 (GLP-1) receptor agonists such as liraglutide, exenatide, lixisenatide, albiglutide, dulaglutide, taspoglutide, semaglutide, and derivatives and combinations thereof. The peptidic active agent may include GLP-1 receptor agonists and/or derivatives thereof. The derivatives may be those disclosed in EP 3 479 841 A2, which is incorporated by reference as if fully set forth herein. Improving therapy adherence is particularly useful for somatostatin receptor and GLP-1 receptor agonists because the patient must administer these active agents very regularly for a satisfactory effect.

Method of Treatment

In an aspect, the invention includes a method of treatment of a patient, comprising administering to said patient an effective amount of an active agent in a composition according to this disclosure.

The method includes enhancing absolute and/or relative oral bioavailability of an active agent.

The invention includes a pharmaceutical composition as described herein for use in therapy.

Method of Making

In an aspect, the invention includes a method of making a pharmaceutical composition, comprising the steps of
a. reacting at least one CPP (cell penetrating peptide) with at least one lipid to obtain a lipid conjugate,
b. optionally purifying the lipid conjugate to obtain a purified lipid conjugate,
c. optionally lyophilizing the lipid conjugate to obtain a lipid conjugate lyophilisate,
d. adding active agent,
e. incorporating lipid conjugate and active agent into a pharmaceutical dosage form,
   wherein the amount of lipid conjugate is from 0.1 to 100 mol % relative to the total amount of lipid in the composition.

The method of this invention may include the step of lyophilizing the lipid conjugate to obtain a lyophilisate. Preferred lyophilisates have limited water content, preferably of not more than 5 wt.-%, or less than 3 wt.-%. The water content can be determined by Karl-Fischer-titration or automated systems such as Water Content Analyzer. Compared to liquid formulations lyophilized lipid conjugates have better long-term stability.

The step of lyophilizing the lipid conjugate may include:
c1. preparing a mixture of the lipid conjugate and at least one lyoprotector.

There is an optimum amount range for lyoprotector in relation to the amount of lipid conjugate in the mixture. Preferably, the amount of lyoprotector ranges from 0.01 to 2 g lyoprotector per gram of lipid conjugate, preferably from 0.02 to 1 g per gram of lipid conjugate, or from 0.03 to 0.5 g per gram of lipid conjugate. In preferred embodiments, a minimum value is at least 0.03 g per 1 g of lipid conjugate. A maximum value may be 0.3, 0.2 or 0.1 g lyoprotector per g of lipid conjugate. In embodiments, these limitations concerning the amount of lyoprotector relative to the amount of lipid apply to the pharmaceutical composition.

Alternatively or in addition, the active agent may be lyophilized. In an embodiment, the pharmaceutical composition comprises a mixture of lyophilized lipid conjugate and lyophilized active agent.

The lyoprotector may be selected from the saccharides, preferably monosaccharides or disaccharides, including sugars and sugar alcohols. The lyoprotector may be selected from sucrose, mannitol, glucose, trehalose, lactose, palatinose and combinations thereof.

The step of incorporating lipid conjugate and active agent into a pharmaceutical dosage form may include, without limitation, the preparation of tablets, pills, capsules, pellets, liquids (including suspensions), powder, effervescent formulations, pastes, lozenges, chewing gums, gels, sprays or granules.

### BRIEF DESCRIPTION OF FIGURES

FIG. 1A Chemical structure of activated lipid Tfp-PEG₁₃-DSPE.

FIG. 1B Chemical structure of activated lipid Mal-PEG₁₂-DSPE.

FIG. 2 Diagram of particle size and PDI of lipid conjugates and octreotide formed in citrate buffer, and changes of size and PDI over time

FIG. 3 Diagram of zeta potential measured at 10 minutes, 3 hours and 24 hours after preparation of particles

FIG. 4 Diagram of absolute oral bioavailability of octreotide in compositions with lipid conjugate, without lipid conjugate and in CPP-lipid liposomes

FIG. 5 Plasma concentration curves and resulting diagram of absolute oral bioavailability of octreotide in compositions with lipid conjugate and without lipid conjugate

FIG. 6 Plasma concentration curves and resulting diagram of absolute oral bioavailability of pasireotide in compositions with lipid conjugate and without lipid conjugate

FIG. 7 Plasma concentration curves of pasireotide after administration of active agent with (black symbols) and without lipid conjugates (white symbols)

FIG. 8 Synthesis of exemplary conjugates

FIG. 9 Synthesis of exemplary conjugates

FIG. 10 Structure of a modified phospholipid used to make a conjugate.

FIG. 11 Example conjugate and modified phospholipid

FIG. 12 Further example conjugate and modified phospholipid

FIG. 13 Comparison of zetapotentials of both octreotide and exenatide containing SEDDS

FIG. 14 Comparison of particles sizes of both octreotide and exenatide containing SEDDS

FIG. 15 Comparison of PDI values of both octreotide and exenatide containing SEDDS

FIG. 16 Example of a conjugated lipid

FIG. 17 Diagrams of size, PDI and zetapotential of micelles containing a conjugated lipid

FIG. 18 Diagram of absolute oral bioavailability of octreotide in compositions with lipid conjugate SEDDS and without lipid conjugate

FIG. 19 Diagram of absolute oral bioavailability of exenatide in compositions with lipid conjugate SEDDS, lipid conjugate micelles and without lipid conjugate.

### EXAMPLES

Procedures

Zetasizer Measurements

The particle size, PDI and zeta potential were determined at room temperature using a zetasizer Nano ZS from Malvern™ (Malvern Instruments Ltd., Worcestershire, United Kingdom). Size and PDI were measured after dilution to a lipid concentration of 0.07 mg/ml with a 10 mM phosphate buffer with a pH of 7.4 using the automatic mode. The zeta potential was determined after dilution to a lipid concentration of 0.14 mg/ml by a 50 mM phosphate buffer with a pH of 7.4. The default settings of the automatic mode of the zetasizer Nano ZS from Malvern™ (Malvern Instruments Ltd., Worcestershire, United Kingdom) were the following: number of measurements=3; run duration=10 s; number of runs=10; equilibration time=60 s; refractive index solvent 1.330; refractive index polystyrene cuvette 1.590; viscosity=0.8872 mPa s; temperature=25° C.; dielectric constant=78.5 F/m; backscattering mode (173°); automatic voltage selection; Smoluchowski equation.

1. Conjugate Synthesis

The synthesis of the lipid conjugate consists of the solid-phase peptide synthesis of lysinyl-nona-arginine utilizing the Fmoc/tBu strategy. Purity is controlled after loading of lysine on a solid phase resin, after coupling of five arginines, and after completion of the solid-phase synthesis. The peptide is cleaved from the resin with side-chain protection groups intact using HFIP/DCM and purified via HPLC. The head-to-tail cyclization of the side-chain protected peptide is performed in solution (ACN/DCM) using HATU/DI EA, which impedes racemization, followed by deprotection with TFA/water/anisole and precipitation with MTBE to obtain the cyclo(Arg-Arg-Arg-Arg-Arg-Arg-Arg-Arg-Arg-Lys) as intermediate. Purification of the peptide intermediate is performed by HPLC on a Phenyl/Hexyl column.

The peptide intermediate is conjugated in solution (DMF/water) with 1.2 equivalents of the second intermediate (2R)-3-((((4,46-dioxo-46-(2,3,5,6-tetrafluorophenoxy)-7,10,13,16,19,22,25,28,31,34,37,40,43-tridecaoxa-3-azahexatetracontyl)oxy)(hydroxy)-phosphoryl)oxy)propane-1,2-diyldistearate and purified via HPLC on a Phenyl/Hexyl column with concurrent ion exchange to acetic acid.

The resulting lipid conjugate is shown in FIG. 11.

2. Synthesis of the Lipid Conjugates

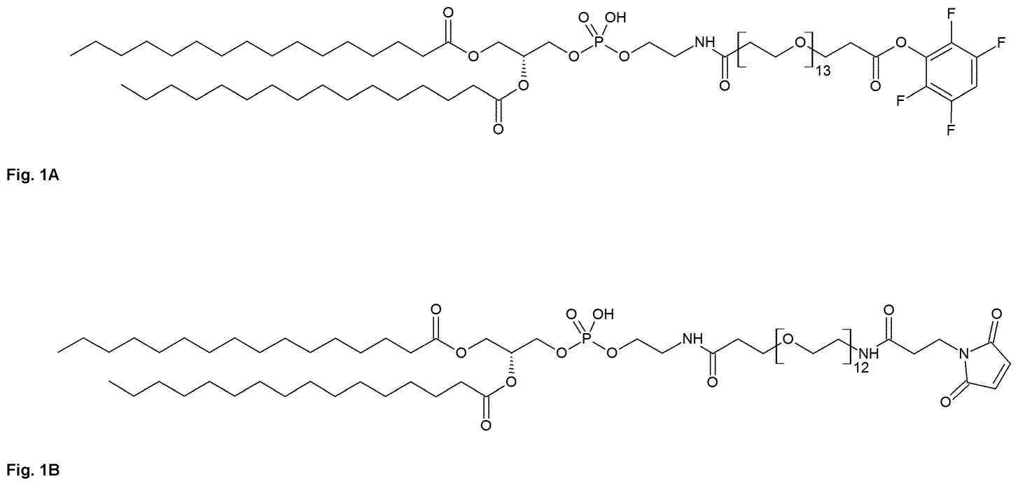

3 equivalents of CPP (cyclic R9-peptides) and 10 equivalents of DIPEA were added to maleimido-PEG(12)-distearoylphosphatidylethanolamine (Iris Biotech) (shown in FIG. 1B) or PEG(13)-distearoylphosphatidylethanolamine-tetrafluorophenyl ester (Iris Biotech) (shown in FIG. 1A) dissolved in DMF in a concentration of 5 mg/ml. The reaction mixture was stirred overnight at room temperature. The reaction mixture was diluted with a 1:2 mixture of ACN/H₂O and purification was performed via HPLC using a Chromolithe® Performance RP-C18e column (100×3 mm). Water and acetonitrile containing 0.05% TFA were used as eluents with a flow rate of 2 ml/min.

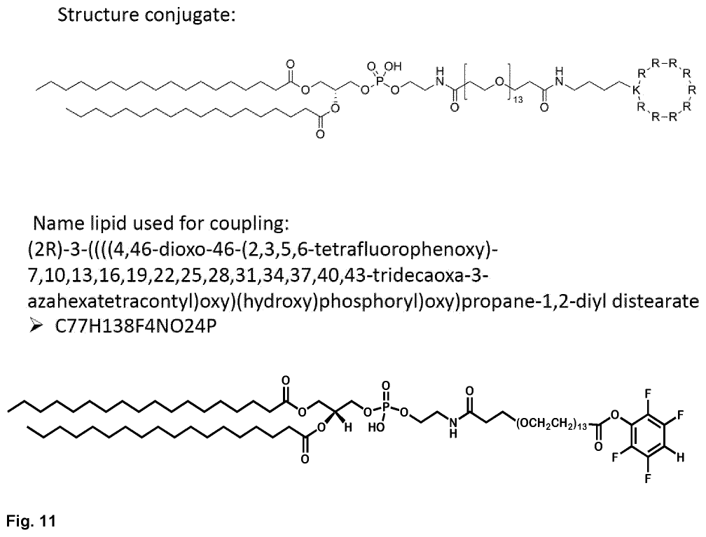

FIG. 12 shows the resulting lipid conjugate structure.

3. Administration of Octreotide to Beagle Dogs

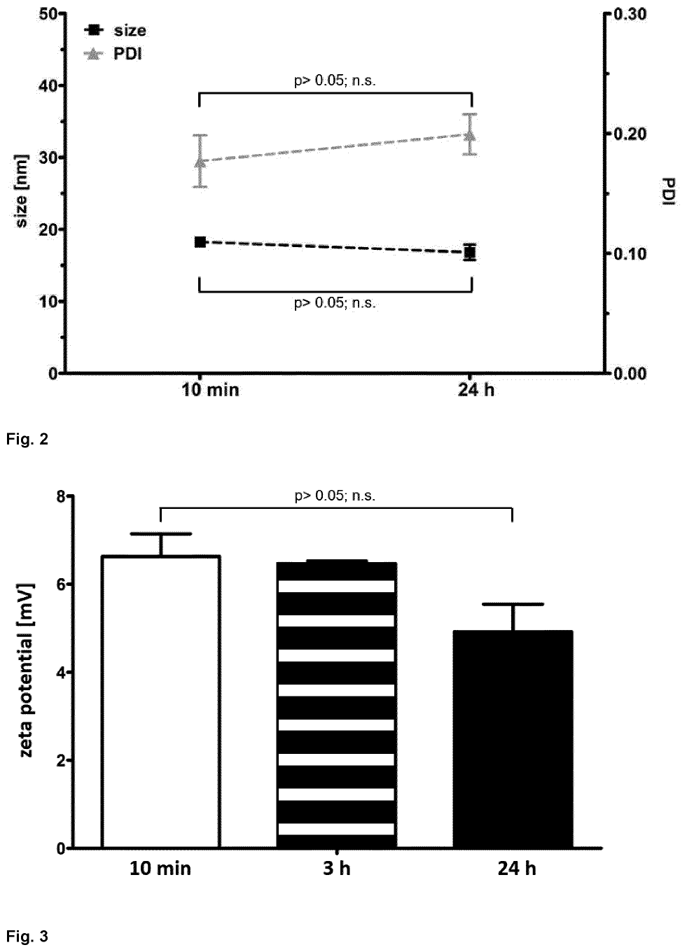

The lipid conjugate produced according to example 2 was used in lyophilized form. The lyophilized lipid conjugate was blended with lyophilized octreotide and suspended in citrate buffer. Upon suspension, the lipid conjugate formed particles. The particles had sizes of about 10 to 20 nm with a PDI in the range of from 0.17 to 0.20 (FIG. 2). Particles were remarkably stable over a period of 24 hours. FIG. 3 shows that the zeta potential of the particles was stable over 24 hours. The zeta potential was in a range of about 3.5 mV to about 7.0 mV.

A bioavailability study was performed at LPT, Hamburg, Germany (Study No 36229) by analyzing the systemic absolute bioavailability of octreotide. The pharmaceutical composition comprising 5 mg lipid conjugate and 1.5 mg octreotide, prepared in citrate buffer (100 mM, pH 5.5) was administered to four beagle dogs by gavage. The amount of active agent in plasma was measured and compared to administration of active agent alone in a dose of 1.5 mg, and with active agent in CPP-lipid-conjugate liposomes prepared in citrate buffer (100 mM, pH 5.5) according to WO 2018/178395 A1.

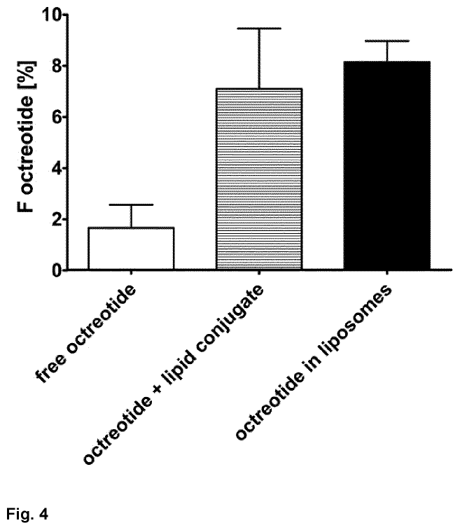

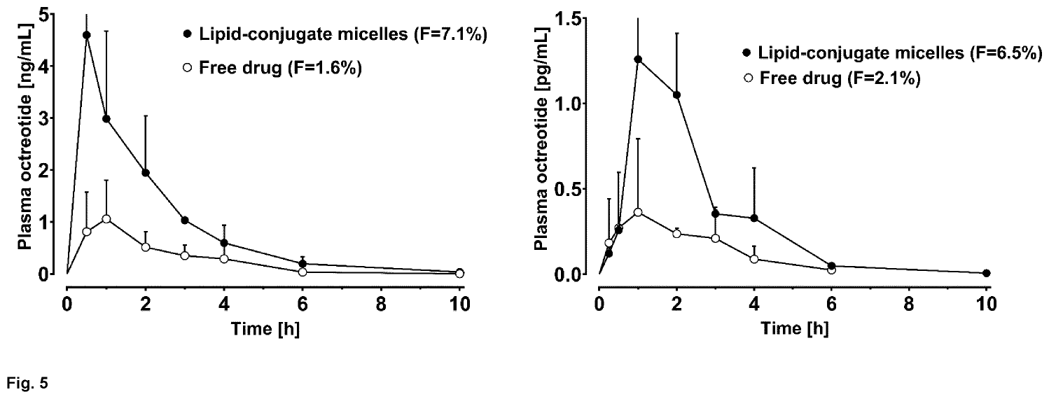

The results are shown in FIGS. 4 and 5. FIG. 5 illustrates the plasma concentration curves and the resulting absolute bioavailabilities. The absolute bioavailability of oral octreotide increased more than 4-fold due to the lipid-conjugate in the composition. The absolute bioavailability, i.e. compared to intravenous bolus administration of 0.1 mg/dog, was essentially the same as bioavailability of the same active agent in the liposome.

4. Administration of Pasireotide to Beagle Dogs

The experiment of example 3 was repeated with 1.5 mg pasireotide as active agent, comparing the active agent in citrate buffer (100 mM, pH 5.5) with and without 5 mg of lipid conjugate.

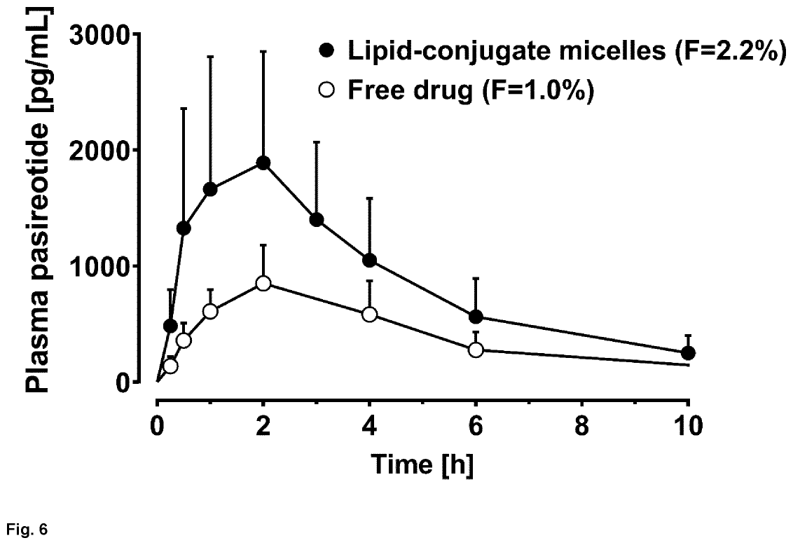

The result is shown in FIG. 6. The bioavailability was more than doubled due to the lipid conjugate in the composition.

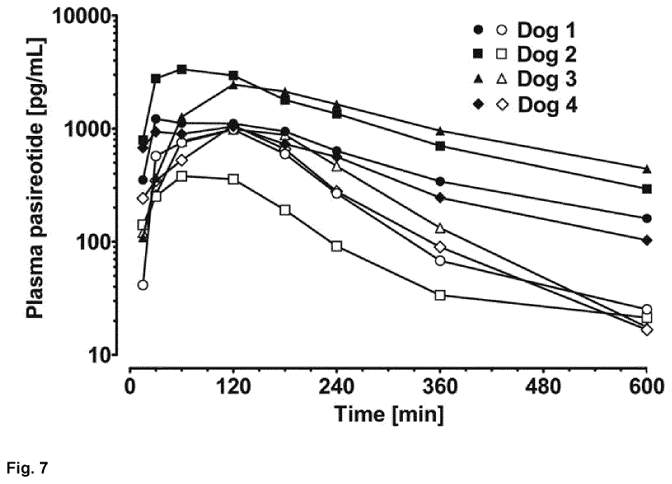

FIG. 7 shows the pharmacokinetics of pasireotide in plasma of beagle dogs after administration of free active agent (white symbols) and active agent with lipid conjugate (black symbols).

5. Pharmacokinetic Evaluation of Octreotide Lipid Conjugate

The effect of lipid conjugate as absorption enhancer for octreotide was investigated in a study in beagle dogs at Charles River, Evreux, France (Study No 47656 PAC), by analyzing the systemic absolute bioavailability of octreotide. Animals were treated with a combination of octreotide/lipid conjugate or octreotide alone, prepared in citrate buffer (100 mM, pH 5.5), by single oral administration via gavage (table). For determination of the absolute bioavailability plasma concentrations after intravenous bolus injection were determined. Between the cycles, a washout phase of one week was performed.

The following observations were included in the overall study design:

Body weight, food consumption, and clinical observations:

Morbidity and mortality: each animal was checked for mortality and morbidity at least once a day during the study, including weekends and public holidays. Clinical signs: each animal was observed at least once a day, during pre-treatment and on the day of treatment, for the recording of clinical signs.

No signs of toxicity were observed throughout the study.

First, animals received an intravenous bolus injection of 0.01 mg/kg octreotide. The consecutive administrations were given orally by gavage. After a wash-out of one week animals #1 and #2 received 0.12 mg/kg free octreotide and animals #3 and #4 received 0.12 mg/kg octreotide with 0.3 mg/kg lipid-conjugate. After a wash-out of one week animals #1 and #2 received 0.12 mg/kg free octreotide with 1 mg/kg lipid-conjugate and animals #3 and #4 received 0.12 mg/kg octreotide with 3 mg/kg lipid-conjugate. After a wash-out of one week animals #1 and #2 received 0.12 mg/kg free octreotide with 10 mg/kg lipid-conjugate and animals #3 and #4 received 0.12 mg/kg octreotide with 30 mg/kg lipid-conjugate. After a wash-out of one week animals #1 and #2 received 0.012 mg/kg free octreotide with 1 mg/kg lipid-conjugate and animals #3 and #4 received 1.2 mg/kg octreotide with 1 mg/kg lipid-conjugate.

The lipid conjugate increased bioavailability of oral octreotide up to more than 3-fold. With respect to the determined bioavailability of octreotide and to the efforts to use the minimal required amount of lipid conjugate for the desired formulation, a dose range of 0.3-1.0 mg/kg conjugate is considered to be the optimal dose. Effective octreotide plasma concentrations were reached with 0.12 mg/kg octreotide. The respective calculated absolute bioavailabilities are listed in the table below. Additionally, a 10-fold increase in octreotide dose (1.2 mg/kg) revealed approximate dose linearity when administered with 1 mg/kg lipid-conjugate.

| | | | | | | |
|---|---|---|---|---|---|---|
| conjugate dose [mg] | 0 | 3 | 10 | 30 | 100 | 300 |
| abs. bioavailability [%] | 2.1 | 4.3 | 6.5 | 6.1 | 3.4 | 6.3 |

6. Preparation of Conjugates

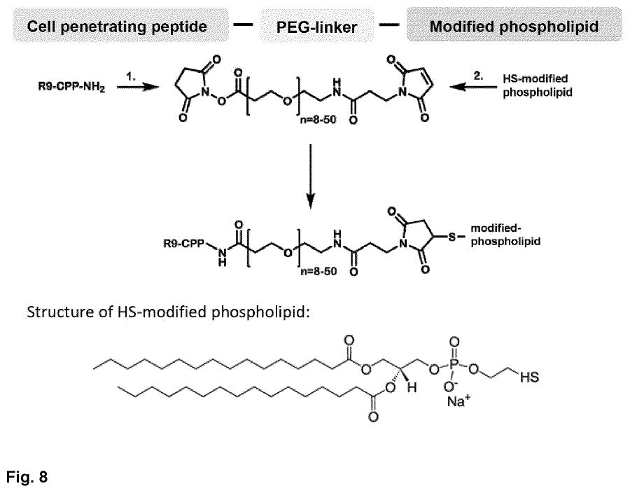

FIG. 8 illustrates the synthesis of exemplary conjugates. The linker used in this example was SM(PEG)8, a PEGylated, long-chain SMCC crosslinker, and succinimidyl([N-maleimidopropionamido]-ethyleneglycol)ester, respectively. R9-CPP stands for either the linear or cyclic nona-arginine peptide. In case of the cyclic it is cyclized via a lysine (R9K) and coupled at the side chain amino function of this lysine.

For coupling of cyclic CPP to the bifunctional PEG-linker, as a first step the cyclized CPP was coupled by an additional lysine to the linker (1.). For this reaction an excess of the CPP was used. In the second step this intermediate product was coupled to the thiol modified phospholipid (2.). The modified phospholipid was 1,2-dipalmitoyl-sn-glycero-3-phosphothioethanol, sodium salt.

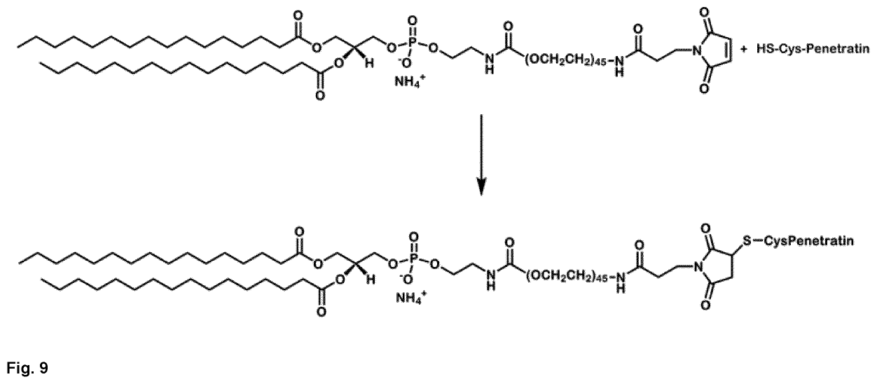

FIG. 9 illustrates the synthesis of exemplary conjugates.

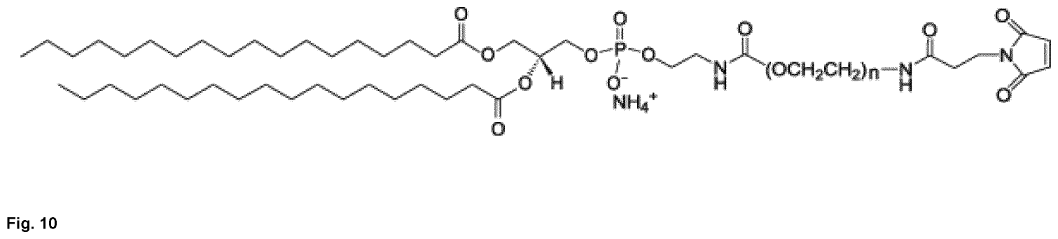

Cysteine-modified penetratin was coupled to the headgroup-modified phospholipid which is shown in FIG. 10. Its chemical name is 1,2-distearoyl-sn-glycero-3-phosphoethanolamine-N-[maleimide(polyethylene glycol)-2000], ammonium salt.

FIG. 11 shows an exemplary conjugate and a modified phospholipid used to make the conjugate.

A further conjugate comprising cyclic R9C was prepared. The conjugate prepared and the modified lipid used to make it are shown in FIG. 12.

7. Preparation of SEDDS

SEDDS containing either octreotide (c=0.21 mg/ml) or exenatide (c=0.14 mg/ml) with a lipid conjugate concentration of c=1.5 mg/ml were prepared as follows:

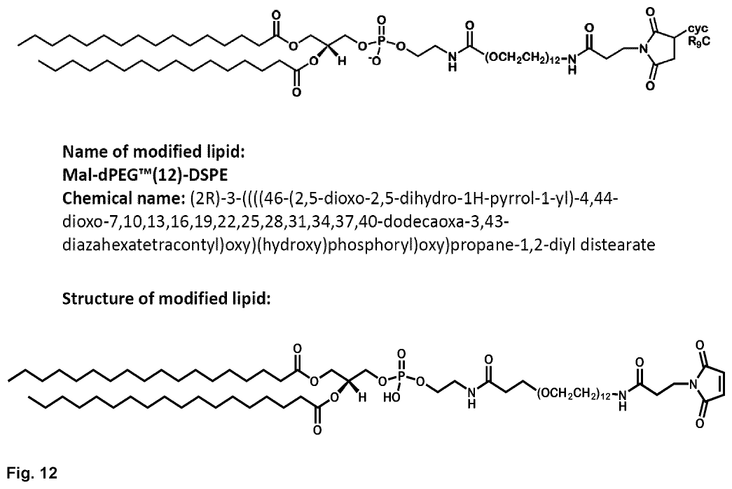

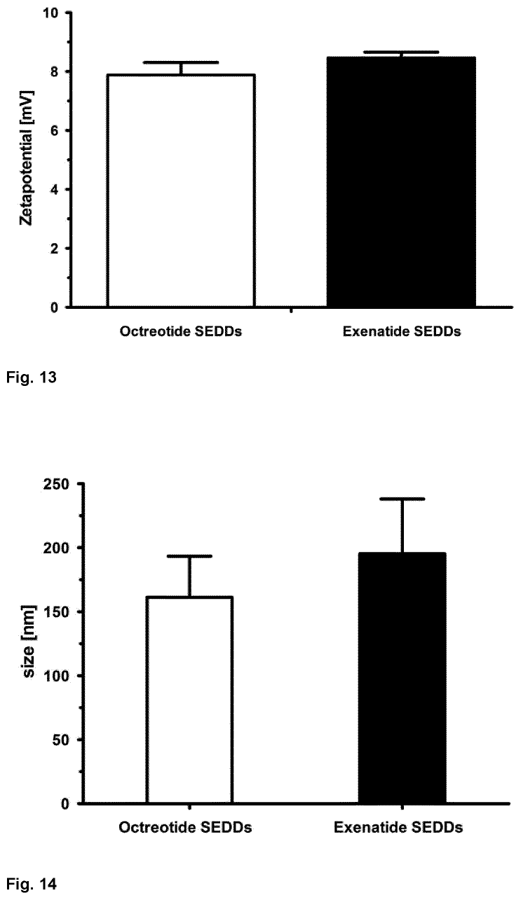

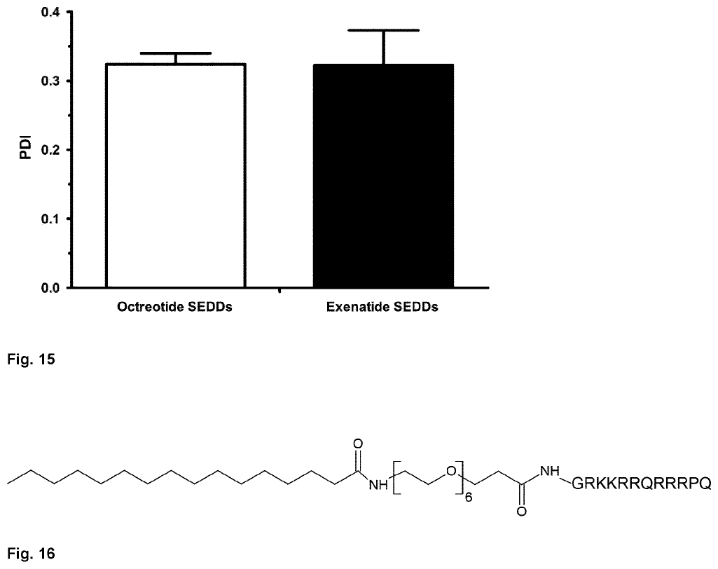

The oily component (corn oil; 310 mg, 10.33 mg/ml) was weighted in a vial. Subsequently, the required amounts of lipid-conjugate (as shown in FIG. 11) and the respective API were added. Afterwards, the SEDDS were prepared by the addition of the required amount of citrate buffer (pH=5.5). Upon suspension, the lipid-conjugate and the oily component formed SEDDS. The SEDDS had sizes of about 150 to 200 nm with a PDI in the range of 0.3 to 0.4 (FIGS. 14 and 15) and a strong positive zeta potential of about +8 mV (FIG. 13). The concentration of octreotide was 0.21 mg/ml, and exenatide concentration was 0.14 mg/ml. The number of measurements was n=3 for zeta potential and PDI, n=6 for particle size.

The encapsulation efficiency was 98.06% for octreotide and 67.55% for exenatide. The encapsulation efficiency of SEDDS containing either octreotide (c=0.21 mg/ml) or exenatide (c=0.14 mg/ml) with a lipid conjugate concentration of c=1.5 mg/ml was determined as follows:

After preparation of SEDDS, a volume of 500 μl of the respective formulation was loaded on a Sephadex G-25 gel filtration column (NAP-5 size exclusion column). Elution was performed with a volume of 1 ml of water. The amount of the respective API in this eluted fraction was compared with the API content of the unpurified fraction under consideration of potential dilution effects. Concentrations were determined by UPLC-MS/MS quantification.

8. Bioavailability of Octreotide SEDDS Formulation

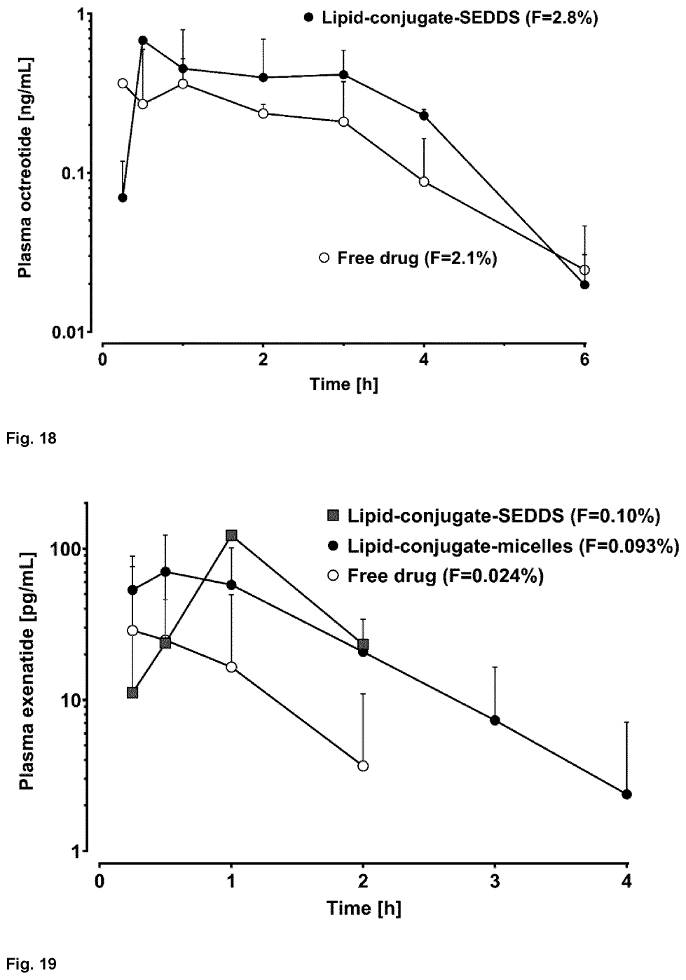

The administration of octreotide in SEDDS formulation was investigated in a study in beagle dogs at Charles River, Evreux, France (Study No 47656 PAC and 48482 PAC). Individual systemic absolute bioavailability of octreotide in SEDDS (dose: 1.5 mg/animal) was evaluated by comparison to intravenous administration of 0.1 mg/animal of free drug, and oral administration of free drug at a dose of 1.5 mg/animal. Animals were treated with octreotide in SEDDs formulation, or octreotide alone, prepared in citrate buffer (100 mM, pH 5.5), by single oral administration via gavage. For determination of the absolute bioavailability plasma concentrations after intravenous bolus injection (in PBS) were determined. Between the cycles, a wash-out phase of one week was performed. Bioavailability of the SEDDS formulation was 2.8%, whereas free drug bioavailability was 2.1% (FIG. 18). Thus, performance of the SEDDS formulation was about 25% improved by the SEDDS.

9. Preparation of C16-TAT Micelles

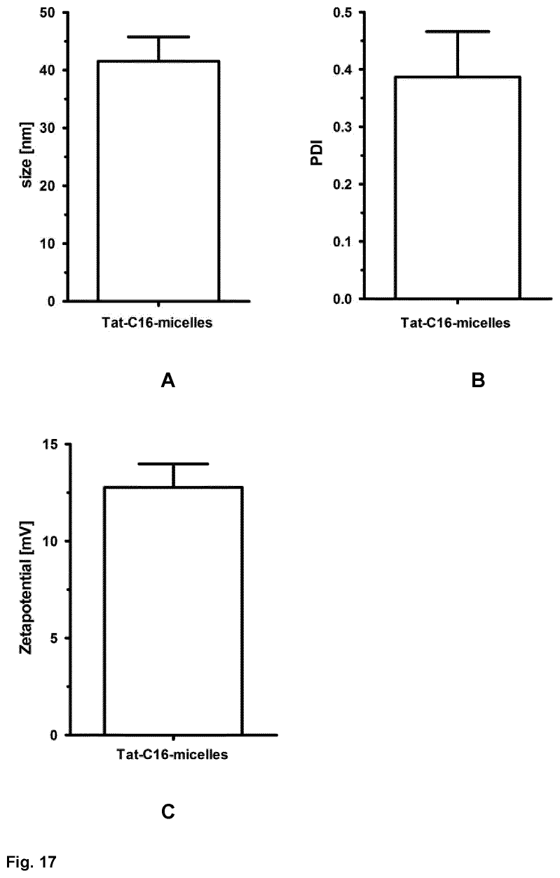

The C16-TAT lipid-conjugate (see FIG. 16) was used in lyophilized form. The lyophilized lipid-conjugate was blended with lyophilized octreotide and suspended in citrate buffer (pH=5.5). Upon suspension, the lipid conjugate formed micelles. The particles had sizes of about 40 to 50 nm with a PDI in the range of from 0.3 to 0.5 (FIGS. 17A and B). The zeta potential was in a range of about +10 mV to about +15 mV (FIG. 17C). FIG. 17 shows the results of measurement of size, PDI and zetapotential of C16-TAT-micelles (c=1 mg/ml), containing 0.2 mg/ml octreotide), n=3.

10. Preparation of Exenatide Formulations

The co-administration of lipid conjugate with the peptide therapeutic exenatide was investigated in a study in beagle dogs at Charles River, Evreux, France (Study No 48482 PAC). Individual systemic absolute bioavailability of exenatide in SEDDS (dose: 1 mg/animal) and micellar formulation (only lipid-conjugate in lyophilized form; dose: 1 mg/animal) was evaluated by comparison to intravenous administration of 0.1 mg/animal of free drug, and oral administration of free drug at a dose of 1 mg/animal. Animals were treated with a combination of exenatide/lipid conjugate, exenatide in SEDDs formulation, or exenatide alone, prepared in citrate buffer (100 mM, pH 5.5), by single oral administration via gavage. For determination of the absolute bioavailability plasma concentrations after intravenous bolus injection (in PBS) were determined. Between the cycles, a wash-out phase of one week was performed. Compared to free drug, the bioavailability of exenatide in a lipid-conjugate micelle formulation with Aprotinin was increased by factor 3.9 (F=0.093%), and in lipid-conjugate SEDDS formulation by a factor of 4.2 (F=0.10%) in beagle dogs (FIG. 19).

The SEDDS were prepared as described in example 7. In the bioavailability study in beagles, each dog received 7 ml of the SEDDS formulation.

For preparation of the micellar formulation, the lipid-conjugate was used in lyophilized form. The lyophilized lipid-conjugate was blended with lyophilized exenatide (c=0.14 mg/ml) and the protease inhibitor Aprotinin (c=3.5 mg/ml) and suspended in citrate buffer (pH=5.5). Upon suspension, the lipid conjugate formed micelles. In the bioavailability study in beagles, each dog received 7 ml of the micelle formulation.

Plasma concentrations of all investigated peptide therapeutics were determined by quantification using a validated UPLC-MS/MS assay (according to the pertinent recommendations for bioanalytical method development of the US FDA and EMA). All assays were performed on a Waters Xevo TQ-XS triple quadrupole tandem mass spectrometer coupled to a Waters Acquity classic UPLC using C18 columns, heated electrospray ionization (ESI), and selected ion monitoring in the positive ion mode.

1. A pharmaceutical composition for oral administration comprising
a conjugate comprising a cell penetrating peptide conjugated to a lipid, and
an active agent,
wherein the composition is essentially free of liposomes.

2. The pharmaceutical composition according to claim 1, wherein the cell penetrating peptide is selected from penetratin, TAT (transactivator of transcription), MAP (model amphiphatic peptide), polyarginines (including R3, R4, R5, R6, R7, R8, R9, R10, R11 and R12), pVEC, transportan, MPG, and functional derivatives, peptide-mimetics and combinations thereof.

3. The pharmaceutical composition according to claim 1, wherein the cell penetrating peptide is a cyclic peptide.

4. The pharmaceutical composition according to claim 1, wherein oral bioavailability of the active agent is increased by at least 50% compared to oral bioavailability of the same composition without the conjugate.

5. The pharmaceutical composition according to claim 1, wherein absolute oral bioavailability of the active agent in the composition is at least 2.5%.

6. The pharmaceutical composition according to claim 1, wherein the active agent is a peptide, such as a cyclic peptide.

7. The pharmaceutical composition according to claim 1, wherein the composition is liquid, solid, or semi-solid.

8. The pharmaceutical composition according to claim 1,
wherein the amount of conjugated lipid is from 0.1 to 100 mol % relative to the total amount of oily components in the composition,
wherein the amount of conjugated lipid is from 5.0 to 100 mol % relative to the total amount of oily components in the composition,
wherein the total amount of oily components in the composition is from 0.1 to 100 mol % relative to the total amount of lipid conjugate in the composition, and/or
wherein the total amount of oily components in the composition is from 5.0 to 100 mol % relative to the total amount of lipid conjugate in the composition.

9. The pharmaceutical composition according to claim 1, wherein the active agent exhibits a degradation half-life in simulated gastric and/or intestinal fluid at 37° C. of at least 10 minutes.

10. The pharmaceutical composition according to claim 1, wherein
the lipid is a phospholipid, optionally selected from phosphatidylcholines, phosphatidylethanolamines, phosphatidylinosites, phosphatidylserines, cephalines, phosphatidylglycerols, lysophospholipids, and combinations thereof, and/or
the lipid is selected from the group consisting of steroids (including cholesterol and its derivatives), fatty acids, fatty alcohols, fatty amines, hydrocarbons with carbon chain lengths of at least eight carbon atoms, sphingolipids, ceramides, glycolipids, etherlipids, carotenoids, glycerides and combinations thereof.

11. The pharmaceutical composition according to claim 1, wherein the composition comprises an aqueous solution with particles therein, wherein the particles comprise the lipid conjugate.

12. The pharmaceutical composition according to claim 11, wherein the particles have a particle size of less than 100 nm, or less than 75 nm.

13. The pharmaceutical composition according to claim 11, wherein the composition is essentially free of liposomes.

14. The pharmaceutical composition according to claim 1, wherein the amount of lipid conjugate in the composition is from 0.1 to 1000 mg/g.

15. A pharmaceutical composition comprising
at least one conjugated lipid comprising a cell penetrating peptide conjugated to a lipid, such as a phospholipid or fatty acid, and
at least one active agent,
wherein the amount of conjugated lipid is from 0.1 to 100 mol % relative to the total amount of oily components in the composition.

16. The pharmaceutical composition according to claim 15, wherein the active agent is selected from the group consisting of peptide, polypeptide and protein.

17. The pharmaceutical composition according to claim 16, wherein the composition does not contain cholesterol in amounts of more than 1.0 mol % relative to the total amount of the oily components.

18. The pharmaceutical composition according to claim 15, wherein the total amount of oily components in the composition comprises the cumulative amounts of steroids (including cholesterol and its derivatives), fatty acids, fatty alcohols, fatty amines, hydrocarbons with carbon chain lengths of at least eight carbon atoms, phospholipids, sphingolipids, ceramides, glycolipids, etherlipids, polyethers, carotenoids, and glycerides (mono-, di- and/or triglycerides) and combinations thereof, including modified mono-, di- or triglycerides and/or modified fatty acids.

19. The pharmaceutical composition according to claim 15, wherein the oily components are the components of the composition that are immiscible with water at 25° C.

20. The pharmaceutical composition according to claim 15, wherein the oily components are components having saturated or unsaturated carbon chain lengths of more than 6, more than 8 or more than 10 carbon atoms.

21. The pharmaceutical composition according to claim 15, wherein the amount of conjugated lipid is from 0.1 to 100 mol %, or from 5.0 to 100 mol %, of the total lipid content in the composition, wherein the total lipid content is the sum of the proportions of conjugated lipid and oily component in the composition.


SEQUENCE LISTING (9 sequences)

SEQ ID NO: 1 - protein, 16 aa, Drosophila melanogaster
RQIKIWFQNRRMKWKK

SEQ ID NO: 2 - protein, 16 aa, Human immunodeficiency virus type 1
CGRKKKRRQRRRPPQC

SEQ ID NO: 3 - protein, 27 aa, Artificial Sequence
  artificially designed cell penetrating peptide
GALFLGFLGAAGSTMGAWSQPKSKRKV

SEQ ID NO: 4 - protein, 9 aa, Artificial Sequence
  artificially designed cell penetrating peptide
RRRRRRRRR

SEQ ID NO: 5 - protein, 18 aa, Mus musculus
  MISC_FEATURE (18)..(18): K-amide
LLIILRRRIRKQAHAHSK

SEQ ID NO: 6 - protein, 28 aa, Homo sapiens
  MISC_FEATURE (28)..(28): L-amide
GWTLNSAGYLLGKINLKALAALAKISIL

SEQ ID NO: 7 - protein, 27 aa, Human immunodeficiency virus
GALFLGFLGAAGSTMGAWSQPKSKRKV

SEQ ID NO: 8 - protein, 10 aa, Artificial Sequence
  artificially designed cell penetrating peptide
RRRRRRRRRC

SEQ ID NO: 9 - protein, 10 aa, Artificial Sequence
  artificially designed cell penetrating peptide
RRRRRRRRRK